(19) United States
(12) Patent Application Publication
(10) Pub. No.: US 2013/0296278 A1
(43) Pub. Date: Nov. 7, 2013

(54) Diazeniumdiolate Derivatives

(71) Applicants: Amjad Ali, Freehold, NJ (US); Michael Man-Chu Lo, Edison, NJ (US); Edward Metzger, Somerset, NJ (US); Shrenik K. Shah, Metuchen, NJ (US); James Dellureficio, Scotch Plains, NJ (US)

(72) Inventors: Amjad Ali, Freehold, NJ (US); Michael Man-Chu Lo, Edison, NJ (US); Edward Metzger, Somerset, NJ (US); Shrenik K. Shah, Metuchen, NJ (US); James Dellureficio, Scotch Plains, NJ (US)

(73) Assignee: Merck Sharp & Dohme Corp., Rahway, NJ (US)

(21) Appl. No.: 13/980,442

(22) PCT Filed: Jan. 12, 2012
(86) PCT No.: PCT/US12/21048

§ 371 Date: Jul. 18, 2013

Related U.S. Application Data
(60) Provisional application No. 61/434,148, filed on Jan. 19, 2011.

(51) Int. Cl. C07C 291/02

(52) U.S. Cl. CPC C07C 291/02
USPC 514/151; 534/556

(57) ABSTRACT

A compound having the structure (I) useful for treating hypertension, pulmonary arterial hypertension, congestive heart failure, conditions resulting from excessive water retention, cardiovascular disease, diabetes, oxidative stress, endothelial dysfunction, cirrhosis, pre-eclampsia, osteoporosis or nephropathy.

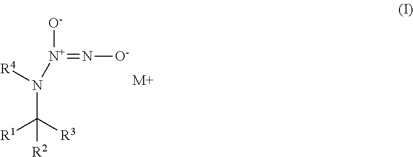

### BACKGROUND OF THE INVENTION

WO09103875 describes diazeniumdiolate dihydro indole derivatives of a specified formula for treating hypertension and cardiovascular disease. WO07144512 describes diazeniumdiolate tetrazole-biphenyl derivatives of a specified formula for treating hypertension and cardiovascular disease. US 2005137191 describes nitrate ester compounds, e.g., 1,2-dichloro-4-(2-methyl-butyldisulfanyl)-benzene, useful for preventing or mitigating tissue and/or cellular damage associated with aging, septic shock, ulcers, gastritis, ulcerative colitis and Crohn's disease. US 2005065194 describes use of an endothelial gene differentiation receptor modulator such as 1-(2-ethoxyphenyl)-3-(hydroxyphenylamino)-pyrrolidine-2,5-dione, to modulate receptor-mediated biological activity such as cell proliferation stimulated by lysophosphatidic acid leading to ovarian cancer and other forms of cancer, and to treat conditions such as cancer, cardiovascular disease, ischemia, and atherosclerosis. WO 9746521 describes aliphatic nitrate esters useful for treating neurological conditions, especially Parkinson's, Alzheimer's and Huntington's disease. WO 2009/0094242 describes angiotensin II receptor antagonists that are prepared with compounds including 1-(N-tert-butylmethylamino)diazen-1-ium-1,2-diolate. Saavedra, et al., J. Med. Chem., 1996, 39, 4361-4365, describes methods for localizing antithrombotic and vasodilatory activity with nitric oxide donors.

Although diazeniumdiolates have significant effects on components of central blood pressure derived indirectly and non-invasively by radial tonometry using pulse wave analysis, there are certain physiological conditions under which diazeniumdiolates are known to release a small amount of corresponding N-nitrosamines as by-products.

The present invention relates to novel diazeniumdiolate derivatives, useful as antihypertensive agents.

### SUMMARY OF THE INVENTION

The present invention includes diazeniumdiolate derivatives, including various pharmaceutically acceptable salts and hydrates of these forms, and pharmaceutical formulations comprising the diazeniumdiolate derivatives. The derivatives are useful as vasodilators for treatment of hypertension, and also as components of such compounds. The compounds advantageously control hypertension by releasing nitric oxide, without forming carcinogenic N-nitrosamines.

The invention also includes a method for treating hypertension, pulmonary arterial hypertension, congestive heart failure, conditions resulting from excessive water retention, cardiovascular disease, diabetes, oxidative stress, endothelial dysfunction, cirrhosis, pre-eclampsia, osteoporosis or nephropathy, comprising administering a compounds of the invention to a patient having such a condition, or being at risk to having such condition.

### DETAILED DESCRIPTION OF THE INVENTION AND PREFERRED EMBODIMENTS

The invention is a compound of formula I:

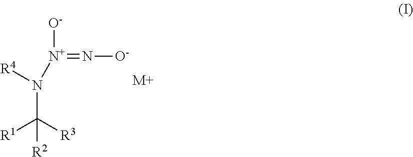

where M+ is a pharmaceutically acceptable cation;
R¹, R² and R³ are independently —C₁₋₆alkyl; and
R⁴ is —C₁₋₆alkyl, —CH₂CH═CH₂, aryl, C₃₋₈carbocycle, heteroaryl, or heterocycle;
wherein alkyl is unsubstituted or independently substituted at any carbon atom with —OH, aryl, C₃₋₈carbocycle, heteroaryl, or heterocycle, and
wherein, when R¹, R² and R³ are —CH₃, R⁴ is not —CH₃ or —CH₂CH₃.

In one embodiment,
R¹, R² and R³ are independently —C₁₋₆alkyl; and
R⁴ is —C₁₋₆alkyl or —CH₂CH═CH₂;
wherein alkyl is unsubstituted or independently substituted at any carbon atom with —OH or —C₆H₅, and
wherein, when R¹, R² and R³ are —CH₃, R⁴ is not —CH₃ or —CH₂CH₃.

In another embodiment, the compound is of formula Ia, which is

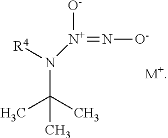

In another embodiment, R⁴ is C₁₋₂alkyl that is substituted with —OH or —C₆H₅.

In another embodiment, R⁴ is unsubstituted C₃₋₆alkyl.

In another embodiment, R⁴ is —CH₂C₆H₅, —CH₂CH₂C₆H₅, —CH₂CH₂OH, —CH₂CH═CH₂, —CH₂CH₂CH₃, —CH₂CH₂CH₂CH₃, —CH₂CH₂CH(CH₃)₂ or —CH₂CH₂C(CH₃)₃.

In another embodiment, the compound is
Sodium 1-(N-tent-butyl-N-propylamino)diazen-1-ium-1,2-diolate,
Sodium 1-(N-tert-butyl-N-allylamino)diazen-1-ium-1,2-diolate,
Sodium 1-[N-tert-butyl-N-(2′-hydroxyethyl)amino]diazen-1-ium-1,2-diolate,
Sodium 1-(N-tent-butyl-N-benzylamino)diazen-1-ium-1,2-diolate,
Sodium 1-(N-tent-butyl-N-butylamino)diazen-1-ium-1,2-diolate,
Sodium 1-[N-tert-butyl-N-(3′-methylbutyl)amino]diazen-1-ium-1,2-diolate,
Sodium 1-[N-tert-butyl-N-(3′,3′-dimethylbutyl)amino]diazen-1-ium-1,2-diolate, or
Sodium 1-[N-tert-butyl-N-(2′-phenylethyl)amino]diazen-1-ium-1,2-diolate.

In another embodiment, the cation is sodium or potassium.

In another embodiment, the cation is sodium.

The invention is also a method for treating hypertension in a patient comprising administering to the patient a compound of formula I:

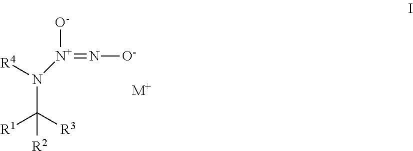

where M+ is a pharmaceutically acceptable cation;
R¹, R² and R³ are independently —C₁₋₆alkyl; and
R⁴ is —C₁₋₆alkyl or —CH₂CH═CH₂;
wherein alkyl is unsubstituted or substituted with —OH or —C₆H₅, and pharmaceutically acceptable salts thereof.

In one embodiment of the method, the compound is of formula Ia, which is

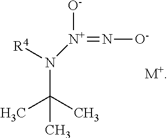

In another embodiment of the method, R₄ is C₁₋₂alkyl that is substituted with —OH or —C₆H₅.

In another embodiment of the method, R⁴ is unsubstituted C₃₋₆alkyl.

In another embodiment of the method, R⁴ is —CH₂C₆H₅, —CH₂CH₂C₆H₅, —CH₂CH₂OH, —CH₂CH═CH₂, —CH₂CH₂CH₃, —CH₂CH₂CH₂CH₃, —CH₂CH₂CH(CH₃)₂ or —CH₂CH₂C(CH₃)₃.

In another embodiment of the method, the compound is
Sodium 1-(N-tert-butyl-N-propylamino)diazen-1-ium-1,2-diolate,
Sodium 1-(N-tert-butyl-N-allylamino)diazen-1-ium-1,2-diolate,
Sodium 1-[N-tert-butyl-N-(2′-hydroxyethyl)amino]diazen-1-ium-1,2-diolate,
Sodium 1-(N-tort-butyl-N-benzylamino)diazen-1-ium-1,2-diolate,
Sodium 1-(N-tert-butyl-N-butylamino)diazen-1-ium-1,2-diolate,
Sodium 1-[N-tert-butyl-N-(3′-methylbutyl)amino]diazen-1-ium-1,2-diolate,
Sodium 1-[N-tert-butyl-N-(3′,3′-dimethylbutyl)amino]diazen-1-ium-1,2-diolate, or
Sodium 1-[N-tert-butyl-N-(2′-phenylethyl)amino]diazen-1-ium-1,2-diolate.

In another embodiment, the cation is sodium or potassium.

In another embodiment, the cation is sodium.

Compounds of the invention can be used to treat hypertension, treat angina, improve insulin sensitivity, and provide renal protection. The compounds can be used alone or in combination (e.g., separate but co-administered, or administered in a fixed dose) with other antihypertensives such as, for example, angiotensin II receptor blockers, diuretics, ACE inhibitors, β-blockers, and calcium channel blockers.

Pharmaceutically acceptable salts include non-toxic salts such as those derived from inorganic acids, e.g. hydrochloric, hydrobromoic, sulfuric, sulfamic, phosphoric, nitric and the like, or the quaternary ammonium salts which are formed, e.g., from inorganic or organic acids or bases. Examples of acid addition salts include acetate, adipate, alginate, aspartate, benzoate, benzenesulfonate, bisulfate, butyrate, citrate, camphorate, camphorsulfonate, carbonate, cyclopentanepropionate, digluconate, dodecylsulfate, ethanesulfonate, fumarate, glucoheptanoate, gluconate, glycerophosphate, hemisulfate, heptanoate, hexanoate, hippurate, hydrochloride, hydrobromide, hydroiodide, 2-hydroxyethanesulfonate, lactate, lactobionate, laurylsulfate, malate, maleate, mesylate, methanesulfonate, 2-naphthalenesulfonate, nicotinate, nitrate, oleate, oxalate, pamoate, pectinate, persulfate, 3-phenylpropionate, picrate, pivalate, propionate, stearate, succinate, sulfate, tartrate, thiocyanate, tosylate, and undecanoate. Additional specific anionic salts include ascorbate, gluceptate, glutamate, glucuronate, besylate, caprylate, isetionate, gentisate, malonate, napasylate, edfisylate, pamoate, xinafoate, and napadisylate.

Base salts include ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine, lysine, and so forth. Also, the basic nitrogen-containing groups may be quaternized with such agents as lower alkyl halides, such as methyl, ethyl, propyl, and butyl chloride, bromides and iodides; dialkyl sulfates like dimethyl, diethyl, dibutyl; and diamyl sulfates, long chain halides such as decyl, lauryl, myristyl and stearyl chlorides, bromides and iodides, aralkyl halides like benzyl and phenethyl bromides and others. Additional specific cationic salts include tromethamine, benzathine, benethamine, diethylammonium, epolamine, hydrabamine. In one embodiment of the invention, the salt is a sodium salt.

When the compounds of the invention contain one chiral center, the term “stereoisomer” includes both enantiomers and mixtures of enantiomers, such as the specific 50:50 mixture referred to as the racemic mixture. The compounds of the present invention may have multiple chiral centers, providing for multiple stereoisomers. This invention includes all of the stereoisomers and mixtures thereof. Unless specifically mentioned otherwise, reference to one stereoisomer applies to any of the possible stereoisomers. Whenever the stereoisomeric composition is unspecified, all possible stereoisomers are included. Where used, the structure marking “*” indicates the location of a carbon atom that is a chiral center. When bonds to a chiral carbon are depicted as straight lines, it is understood that both (R) and (S) configurations of the chiral carbon, and hence both enantiomers and mixtures thereof, are represented.

Some of the compounds described herein may exist as tautomers. The individual tautomers as well as mixtures thereof are encompassed with the described compounds.

As used herein except where noted, “alkyl” is intended to include both branched- and straight-chain saturated aliphatic hydrocarbon groups having the specified number of carbon atoms. Commonly used abbreviations for alkyl groups are used throughout the specification, e.g. methyl may be represented by conventional abbreviations including “Me” or CH₃ or a symbol that is an extended bond as the terminal group, e.g. [symbol] , ethyl may be represented by “Et” or CH₂CH₃, propyl may be represented by “Pr” or CH₂CH₂CH₃, butyl may be represented by “Bu” or CH₂CH₂CH2CH₃, etc. “C₁₋₄ alkyl” (or “C₁-C₄ alkyl”) for example, means linear or branched chain alkyl groups, including all isomers, having the specified number of carbon atoms. C₁₋₄ alkyl includes n-, iso-, sec- and t-butyl, n- and isopropyl, ethyl and methyl. If no number is specified, 1-4 carbon atoms are intended for linear or branched alkyl groups.

The term “alkylene” refers to any divalent linear or branched chain aliphatic hydrocarbon radical having a number of carbon atoms in the specified range. Thus, for example, “—C₁-C₆ alkylene-” refers to any of the C₁ to C₆ linear or branched alkylenes, and “—C₁-C₄ alkylene-” refers to any of the C₁ to C₄ linear or branched alkylenes. A class of alkylenes of particular interest with respect to the invention is —(CH₂)₁₋₆—, and sub-classes of particular interest include —(CH₂)₁₋₄—, —(CH₂)₁₋₃—, —(CH₂)₁₋₂—, and —CH₂—. Another sub-class of interest is an alkylene selected from the group consisting of —CH₂—, —CH(CH₃)—, and —C(CH3)₂—. Expressions such as “C₁-C₄ alkylene-phenyl” and “C₁-C₄ alkyl substituted with phenyl” have the same meaning and are used interchangeably.

Alkyl groups and alkylene groups may be unsubstituted, or substituted with 1 to 3 substituents on any one or more carbon atoms, with halogen, C₁-C₂₀ alkyl, CF₃, NH₂, —NH(C₁-C₆ alkyl), —N(C₁-C₆ alkyl)₂, NO₂, oxo, CN, N₃, —OH, —OC(O)C₁-C₆ alkyl, —O(C₁-C₆ alkyl), C₃-C₁₀ cycloalkyl, C₂-C₆ alkenyl, C₂-C₆ alkynyl, (C₁-C₆ alkyl)S(O)₀₋₂—, HS(O)₀₋₂—, (C₁-C₆ alkyl)S(O)₀₋₂(C₁-C₆ alkyl)-, —HS(O)₀₋₂(C₁-C₆ alkyl)-, (C₀-C₆ alkyl)C(O)NH—, H₂N—C(NH)—, —O(C₁-C₆ alkyl)CF₃, HC(O)—, (C₁-C₆ alkyl)C(O)—, HOC(O)—, (C₁-C₆ alkyl)OC(O)—, HO(C₁-C₆ alkyl)-, (C₁-C₆ alkyl)O(C₁-C₆ alkyl)-, (C₁-C₆ alkyl)C(O)₁₋₂(C₁-C₆ alkyl)-, HC(O)₁₋₂(C₁-C₆ alkyl)-, (C₁-C₆ alkyl)C(O)₁₋₂—, HOC(O)NH—, (C₁-C₆ alkyl)OC(O)NH—, aryl, aralkyl, heterocycle, heterocyclylalkyl, halo-aryl, halo-aralkyl, halo-heterocycle, halo-heterocyclylalkyl, cyano-aryl, cyano-aralkyl, cyano-heterocycle and cyano-heterocyclylalkyl, where such substitution results in formation of a stable compound.

The term “aryl”, alone or in combination, relates to a phenyl, naphthyl or indanyl group, preferably a phenyl group. The abbreviation “Ph” represents phenyl. The term “carbocycle” (and variations thereof such as “carbocyclic” or “carbocyclyl”) as used herein, unless otherwise indicated, refers to a C₃ to C₈ monocyclic saturated or unsaturated ring. The carbocycle may be attached to the rest of the molecule at any carbon atom which results in a stable compound. Saturated carbocyclic rings are also referred to as cycloalkyl rings, e.g., cyclopropyl, cyclobutyl, etc.

The term “heteroaryl” refers to an unsaturated ring having a specified number of atom members (e.g., 5 or 6-membered), including a specified number of heteroatoms (e.g., 1, 2, 3 or 4 heteroatoms independently selected from N, O or S), e.g., 5-membered rings containing one nitrogen (pyrrole), one oxygen (pyran) or one sulfur (thiophene) atom, 5-membered rings containing one nitrogen and one sulfur (thiazole) atom, 5-membered rings containing one nitrogen and one oxygen (oxazole or isoxazole) atom, 5-membered rings containing two nitrogen (imidazole or pyrazole) atoms, five-membered aromatic rings containing three nitrogen atoms, five-membered aromatic rings containing one oxygen, one nitrogen or one sulfur atom, five-membered aromatic rings containing two heteroatoms independently selected from oxygen, nitrogen and sulfur, 6-membered rings containing one nitrogen (pyridine), or one oxygen (furan) atom, 6-membered rings containing two nitrogen (pyrazine, pyrimidine, or pyridazine) atoms, 6-membered rings containing three nitrogen (triazine) atoms, a tetrazolyl ring; a thiazinyl ring; or coumarinyl. Examples of such ring systems are furanyl, thienyl, pyrrolyl, pyridinyl, pyrimidinyl, indolyl, imidazolyl, triazinyl, thiazolyl, isothiazolyl, pyridazinyl, pyrazolyl, oxazolyl, and isoxazolyl.

The terms “heterocycle” and “heterocyclic” refer to a saturated ring having a specified number of atom members and a specified number of heteroatoms, in which the entire ring system (whether mono- or poly-cyclic) is saturated, e.g., a 4- to 8-membered saturated monocyclic ring or a stable 7- to 12-membered bicyclic ring system which consists of carbon atoms and one or more heteroatoms selected from N, O and S, a 5- or 6-membered heterocyclic ring having 1 or 2 heteroatoms which are N, O or S, etc. Representative examples include piperidinyl, piperazinyl, azepanyl, pyrrolidinyl, pyrazolidinyl, imidazolidinyl, oxazolidinyl, isoxazolidinyl, morpholinyl, thiomorpholinyl, thiazolidinyl, isothiazolidinyl, and tetrahydrofuryl (or tetrahydrofuranyl).

Aryl groups and carbocycles may be unsubstituted, or substituted with 1, 2, or 3 substituents on any one or more available carbon atoms, with halogen, C₁-C₂₀ alkyl, CF₃, NH₂, —NH(C₁-C₆ alkyl), —N(C₁-C₆ alkyl)₂, NO₂, oxo, CN, N₃, —OH, —O(C₁-C₆ alkyl), C₃-C₁₀ cycloalkyl, C₂-C₆ alkenyl, C₂-C₆ alkynyl, HS(O)₀₋₂—, (C₁-C₆ alkyl)S(O)₀₋₂—, (C₁-C₆ alkyl)S(O)₀₋₂(C₁-C₆ alkyl)-, HS(O)₀₋₂(C₁-C₆ alkyl)-, (C₁-C₆ alkyl)S(O)₀₋₂, (C₁-C₆ alkyl)C(O)NH—, HC(O)NH—, H₂N—C(NH)—, —O(C₁-C₆ alkyl)CF₃, (C₁-C₆ alkyl)C(O)—, HC(O)—, (C₁-C₆ alkyl)OC(O)—, HOC(O)—, (C₁-C₆ alkyl)O(C₁-C₆ alkyl)-, HO(C₁-C₆ alkyl)-, (C₁-C₆ alkyl)C(O)₁₋₂(C₁-C₆ alkyl)-, (C₁-C₆ alkyl)C(O)₁₋₂—, HC(O)₁₋₂(C₁-C₆ alkyl)-, (C₁-C₆ alkyl)OC(O)NH—, HOC(O)NH—, aryl, aralkyl, heterocycle, heterocyclylalkyl, halo-aryl, halo-aralkyl, halo-heterocycle, halo-heterocyclylalkyl, cyano-aryl, cyano-aralkyl, cyano-heterocycle and cyano-heterocyclylalkyl, where such substitution results in formation of a stable compound.

Heteroaryl groups and heterocycles may be unsubstituted, or substituted with 1, 2, or 3 substituents on any one or more available carbon atoms, with halogen, C₁-C₂₀ alkyl, CF₃, NH₂, —NH(C₁-C₆ alkyl), —N(C₁-C₆ alkyl)₂, NO₂, oxo, CN, N₃, —OH, —O(C₁-C₆ alkyl), C₃-C₁₀ cycloalkyl, C₂-C₆ alkenyl, C₂-C₆ alkynyl, (C₁-C₆ alkyl)S(O)₀₋₂—, HS(O)₀₋₂—, (C₁-C₆ alkyl)S(O)₀₋₂(C₁-C₆ alkyl)-, HS(O)₀₋₂(C₁-C₆ alkyl)-, (C₁-C₆ alkyl)S(O)₀₋₂—, (C₁-C₆ alkyl)C(O)NH—, HC(O)NH—, H₂N—C(NH)—, —O(C₁-C₆ alkyl)CF₃, HC(O)—, (C₁-C₆ alkyl)C(O)—, (C₁-C₆ alkyl)OC(O)—, HOC(O)—, (C₁-C₆ alkyl)O(C₁-C₆ alkyl)-, HO(C₁-C₆ alkyl)-, (C₁-C₆ alkyl)O—, (C₁-C₆ alkyl)C(O)₁₋₂(C₁-C₆ alkyl)-, HC(O)₁₋₂(C₁-C₆ alkyl)-, (C₁-C₆ alkyl)C(O)₁₋₂, (C₁-C₆ alkyl)OC(O)NH—, HOC(O)NH—, aryl, aralkyl, heterocycle, heterocyclylalkyl, halo-aryl, halo-aralkyl, halo-heterocycle, halo-heterocyclylalkyl, cyano-aryl, cyano-aralkyl, cyano-heterocycle or cyano-heterocyclylalkyl, or independently or additionally substituted with 1 or 2 substituents on any one or more available nitrogen atoms, with C₁-C₂₀ alkyl, oxo, C₃-C₁₀ cycloalkyl, C₂-C₆ alkenyl, C₂-C₆ alkynyl, aryl, —C(O)C₁₋₆ alkyl, —C(O)NHC₁-C₆ alkyl, —C(O) NH₂, —C₁-C₆ alkylC(O)NH₂, C₁-C₆ alkylOC(O)NH₂, or independently or additionally substituted with 1 substituent on any one or more sulfur atoms, with C₁-C₂₀ alkyl, oxo, C₃-C₁₀ cycloalkyl, C₂-C₆ alkenyl, C₂-C₆ alkynyl, aryl, where such substitution results in formation of a stable compound. Substituted heterocyclic rings include cyclic ureas, such as imidazolidin-2-one and tetrahydropyrimidin-2(1H)-one, which rings contain three sequential atoms that are nitrogen, carbon and niotrogen, wherein the carbon atom is substituted with an oxo substituent.

The compounds of the invention are useful for treating hypertension, Pulmonary Arterial Hypertension, congestive heart failure, angina, conditions resulting from excessive water retention, cardiovascular diseases, diabetes, oxidative stress, endothelial dysfunction, cirrhosis, pre-eclampsia, osteoporosis, or nephropathy, comprising administering a compounds of the invention to a patient having such a condition, or being at risk to having such condition

The invention also relates to the use of compounds of the invention for the preparation of a medicament for the treatment and/or prophylaxis of the above-mentioned diseases.

The abovementioned compounds of the invention are also of use in combination with other pharmacologically active compounds comprising angiotensin II receptor antagonists (e.g., losartan, valsartan, candesartan, irbesartan, olmesartan) angiotensin converting enzyme inhibitors (e.g, alacepril, benazepril, captopril, ceronapril, cilazapril, delapril, enalapril, enalaprilat, fosinopril, imidapril, lisinopril, moveltipril, perindopril, quinapril, ramipril, spirapril, temocapril, or trandolapril), neutral endopeptidase inhibitors (e.g., thiorphan and phosphoramidon), aldosterone antagonists, renin inhibitors (e.g. urea derivatives of di- and tri-peptides (See U.S. Pat. No. 5,116,835), amino acids and derivatives (U.S. Pat. Nos. 5,095,119 and 5,104,869), amino acid chains linked by non-peptidic bonds (U.S. Pat. No. 5,114,937), di- and tri-peptide derivatives (U.S. Pat. No. 5,106,835), peptidyl amino diols (U.S. Pat. Nos. 5,063,208 and 4,845,079) and peptidyl beta-aminoacyl aminodiol carbamates (U.S. Pat. No. 5,089,471); also, a variety of other peptide analogs as disclosed in the following U.S. Pat. Nos. 5,071,837; 5,064,965; 5,063,207; 5,036,054; 5,036,053; 5,034,512 and 4,894,437, and small molecule renin inhibitors (including diol sulfonamides and sulfinyls (U.S. Pat. No. 5,098,924), N-morpholino derivatives (U.S. Pat. No. 5,055,466), N-heterocyclic alcohols (U.S. Pat. No. 4,885,292) and pyrolimidazolones (U.S. Pat. No. 5,075,451); also, pepstatin derivatives (U.S. Pat. No. 4,980,283) and fluoro- and chloro-derivatives of statone-containing peptides (U.S. Pat. No. 5,066,643), enalkrein, RO 42-5892, A 65317, CP 80794, ES 1005, ES 8891, SQ 34017, aliskiren ((2S,4S,5S,7S)-N-(2-carbamoyl-2-methylpropyl)-5-amino-4-hydroxy-2,7-diisopropyl-8-[4-methoxy-3-(3-methoxypropoxy)phenyl]-octanamid hemifumarate) SPP600, SPP630 and SPP635), endothelin receptors antagonists, vasodilators, calcium channel blockers (e.g., amlodipine, nifedipine, veraparmil, diltiazem, gallopamil, niludipine, nimodipins, nicardipine), potassium channel activators (e.g., nicorandil, pinacidil, cromakalim, minoxidil, aprilkalim, loprazolam), diuretics (e.g., hydrochlorothiazide), sympatholitics, beta-adrenergic blocking drugs (e.g., propranolol, atenolol, bisoprolol, carvedilol, metoprolol, or metoprolol tartate), alpha adrenergic blocking drugs (e.g., doxazocin, prazocin or alpha methyldopa) central alpha adrenergic agonists, peripheral vasodilators (e.g. hydralazine), lipid lowering agents (e.g., simvastatin, lovastatin, ezetamibe, atorvastatin, pravastatin), metabolic altering agents including insulin sensitizing agents and related compounds including (i) PPAR.gamma. agonists, such as the glitazones (e.g. troglitazone, pioglitazone, englitazone, MCC-555, rosiglitazone, balaglitazone, and the like) and other PPAR ligands, including PPAR.alpha./.gamma. dual agonists, such as KRP-297, muraglitazar, naveglitazar, Galida, tesaglitazar, TAK-559, PPAR.alpha. agonists, such as fenofibric acid derivatives (gemfibrozil, clofibrate, fenofibrate and bezafibrate), and selective PPAR.gamma. modulators (SPPAR.gamma.M's), such as disclosed in WO 02/060388, WO 02/08188, WO 2004/019869, WO 2004/020409, WO 2004/020408, and WO 2004/066963; (ii) biguanides such as metformin and phenformin, and (iii) protein tyrosine phosphatase-1 B (PTP-1B) inhibitors, glipizide, DPP-IV inhibitors such as sitagliptin, vildagliptin, alogliptin, and saxagliptin, which inhibit dipeptidyl peptidase-IV enzyme and which are useful for treating diabetes, or with other drugs beneficial for the prevention or the treatment of the above-mentioned diseases including nitroprusside and diazoxide. Such combination can be achieved by combining two or more active ingredients in a single dosage formulation containing the two or more independent active ingredients, e.g., an angiotensin II receptor antagonist and a compound of the invention, or by concurrent but separate administration of the two or more active ingredients.

The dosage regimen utilizing the compound of the invention is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

Oral dosages of the compounds of the invention, when used for the indicated effects, will range between about 0.0125 mg per kg of body weight per day (mg/kg/day) to about 7.5 mg/kg/day, preferably 0.0125 mg/kg/day to 3.75 mg/kg/day, and more preferably 0.3125 mg/kg/day to 1.875 mg/kg/day. For example, an 80 kg patient would receive between about 1 mg/day and 600 mg/day, preferably 1 mg/day to 300 mg/day, more preferably 25 mg/day to 150 mg/day, and more preferably 5 mg/day to 100 mg/day. A suitably prepared medicament for once a day administration would thus contain between 1 mg and 600 mg, preferably between 1 mg and 300 mg, and more preferably between 25 mg and 300 mg, e.g., 25 mg, 50 mg, 100 mg, 150, 200, 250 and 300 mg. Advantageously, the compound of the invention may be administered in divided doses of two, three, or four times daily. For administration twice a day, a suitably prepared medicament would contain between 0.5 mg and 300 mg, preferably between 0.5 mg and 150 mg, more preferably between 12.5 mg and 150 mg, e.g., 12.5 mg, 25 mg, 50 mg, 75 mg, 100 mg, 125 mg and 150 mg.

The compounds of the invention can be administered in such oral forms as tablets, capsules and granules. The compounds of the invention are typically administered as active ingredients in admixture with suitable pharmaceutical binders as described below. % w/w expresses the weight percent of the indicated composition constituent compared to the total composition. Suitable fillers used in these dosage forms include microcrystalline cellulose, silicified microcrystalline cellulose, dicalcium phosphate, lactose, mannitol, and starch, preferably microcrystalline cellulose, dicalcium phosphate, lactose or mixtures thereof. Suitable binders include hydroxypropyl cellulose, hydroxypropyl methyl cellulose, starch, gelatin, natural sugars such as glucose or beta-lactose, corn-sweeteners, natural and synthetic gums such as acacia, tragacanth or sodium alginate, carboxymethylcellulose, and polyvinyl pyrrolidone. Lubricants used in these dosage forms include sodium oleate, sodium stearate, magnesium stearate, sodium benzoate, sodium acetate, sodium chloride, sodium stearyl fumarate, stearic acid and the like, preferably magnesium stearate. Suitable coating compositions include aqueous dispersion or organic solution of insoluble polymers such as ethyl cellulose, cellulose aetate, cellulose acetate butyrate and acrylate copolymers commercially known as Eudragit®. Plasticizers include triethyl citrate, dibutyl sebacate, dibutyl phthalate, triacetin and castor oil. Antitacking agents include talc, kaolin, colloidal silica or mixtures thereof.

### General Method of Synthesis

The methods for preparing the compounds of this invention are described in the following examples. Starting materials and intermediates are made from known procedures or as otherwise illustrated. Variables are as described above unless otherwise indicated.

Scheme 1 describes a method to prepare diazeniumdiolate salts 1-1 from the amine. The amine is dissolved in an appropriate solvent such as methanol, acetonitrile, tetrahydrofuran, N,N-dimethylformamide, or N-methylpyrrolidinone in the presence of a base of the general formula M⁺A⁻ such as sodium methoxide, sodium tert-butoxide, sodium tert-pentoxide, or sodium trimethylsilanolate, or their potassium equivalents, where M⁺ represents sodium or potassium. The reaction mixture is then stirred in the presence of nitric oxide for an extended period of time, e.g., 24 hours, to afford the diazeniumdiolate salts.

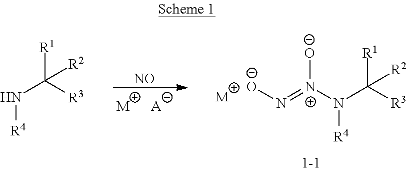

Scheme 2 describes another method to prepare diazeniumdiolate salts from the amine. The amine is dissolved in an appropriate solvent such as methanol, acetonitrile, diethyl ether, tetrahydrofuran, N,N-dimethylformamide, or N-methylpyrrolidinone. The reaction mixture is then stirred in the presence of nitric oxide for an extended period of time, e.g., 24 hours, to afford the diazeniumdiolate salts of structure 2-1. If a metal counterion is desired, the reaction mixture can be charged with a base of the general formula M⁺A⁻, such as sodium methoxide, sodium tert-butoxide, sodium tert-pentoxide, or sodium trimethylsilanolate, or their potassium equivalents, where M⁺ represents sodium or potassium, to afford the diazeniumdiolate salts of structure 2-2.

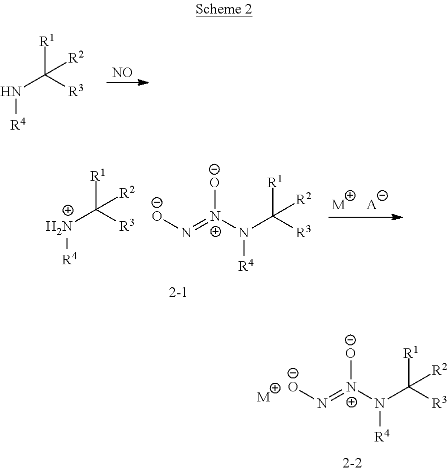

### EXAMPLE 1

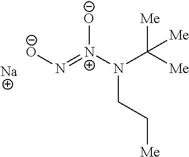 [SMILES: [Na+] ; CCCN(/[N+]([O-])=N/[O-])C(C)(C)C]

### Sodium 1-(N-tert-butyl-N-propylamino)diazen-1-ium-1,2-diolate

To a methanol (2.5 mL) solution of N-tert-butyl-N-propylamine (1.88 g, 16.3 mmol) was added a 25 weight % methanolic solution of sodium methoxide (3.73 mL, 16.3 mmol). The solution was stirred for 24 hours at 25° C. under nitric oxide (350 psi). The methanol was removed in vacuo, and diethyl ether was added to precipitate a white solid. The solid was filtered, washed with diethyl ether, and dried under vacuum to afford the title compound. ¹H NMR (500 MHz, D₂O) δ 2.95 (t, J=7.2 Hz, 2H), 1.25 (sextet, 1=7.4 Hz, 2H), 1.15 (s, 9H), 0.87 (t, J=7.4 Hz, 3H).

### EXAMPLE 2

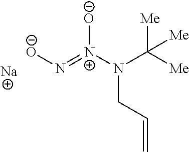

### Sodium 1-(N-text-butyl-N-allylamino)diazen-1-ium-1,2-diolate

The title compound was made by following the procedures described in EXAMPLE 1, substituting N-tert-butyl-N-allylamine for N-tert-butyl-N-propylamine. ¹H NMR (500 MHz, D₂O) δ 5.75 (ddt, J=17.2, 10.1, 6.7 Hz, 1H), 5.23 (dd, J=17.2, 1.7 Hz, 1H), 5.14 (d, J=10.2 Hz, 1H), 3.64 (d, J=6.8 Hz, 2H), 1,21 (s, 9H).

### EXAMPLE 3

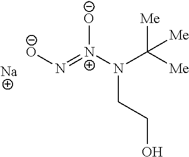 [SMILES: [Na+] ; CC(C)(C)N(CCO)/[N+]([O-])=N/[O-]]

### Sodium 1-[N-tert-butyl-N-(2′-hydroxyethyl)amino]diazen-1-ium-1,2-diolate

The title compound was made by following the procedures described in EXAMPLE 1, substituting 2-(tert-butylamino)ethanol for N-tert-butyl-N-propylamine. ¹H NMR (500 MHz, D₂O) δ 3.50 (t, J=5.8 Hz, 2H), 3.21 (t, 1=5.8 Hz, 2H), 1.21 (s, 9H).

### EXAMPLE 4

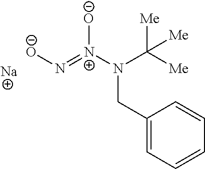 [SMILES: [Na+] ; CC(C)(C)N(Cc1ccccc1)/[N+]([O-])=N/[O-]]

### Sodium 1-(N-tent-butyl-N-benzylamino)diazen-1-ium-1,2-diolate

The title compound was made by following the procedures described in EXAMPLE 1, substituting N-tert-butyl-N-benzylamine for N-tert-butyl-N-propylamine. ¹H NMR (500 MHz, D₂O) δ 7.53 (br s, 5H), 4.24 (s, 2H), 1.50 (s, 9H).

### EXAMPLE 5

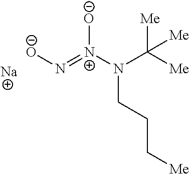 [SMILES: [Na+] ; CCCCN(/[N+]([O-])=N/[O-])C(C)(C)C]

### Sodium 1-(N-tert-butyl-N-butylamino)diazen-1-ium-1,2-diolate

The title compound was made by following the procedures described in EXAMPLE 1, substituting N-tert-butyl-N-butylamine for N-tert-butyl-N-propylamine. ¹H NMR (500 MHz, D₂O) δ 3.09-2.99 (m, 2H), 1.39-1.31 (m, 2H), 1.27 (quintet, J=7.4 Hz, 2H), 1.21 (s, 9H), 0.91 (t, J=7.0Hz, 3H).

### EXAMPLE 6

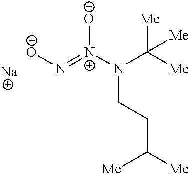 [SMILES: [Na+] ; CC(C)CCN(/[N+]([O-])=N/[O-])C(C)(C)C]

### Sodium 1-[N-tert-butyl-N-(3′-methylbutyl)amino]diazen-1-ium-1,2-diolate

The title compound was made by following the procedures described in EXAMPLE 1, substituting N-tert-butyl-N-(3-methylbutyl)amine for N-tert-butyl-N-propylamine. ¹H NMR (500 MHz, D₂O) δ 3.01 (t, J=7.3 Hz, 2H), 1.66-1.55 (m, 1H), 1.16 (s, 9H), 1.13 (q, J=8.3 Hz, 2H), 0.86 (d, J=6.6 Hz, 6H).

### EXAMPLE 7

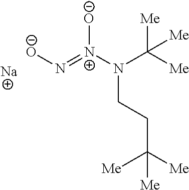 [SMILES: [Na+] ; CC(C)(C)CCN(/[N+]([O-])=N/[O-])C(C)(C)C]

### Sodium 1-[N-tert-butyl-N-(3′,3′-dimethylbutyl)amino]diazen-1-ium-1,2-diolate

The title compound was made by following the procedures described in EXAMPLE 1, substituting N-tert-butyl-N-(3′,3′-dimethylbutyl)amine for N-tert-butyl-N-propylamine. ¹H NMR (500 MHz, D₂O) δ 3.05 (t, J=7.8 Hz, 2H), 1.21 (s, 9H), 1.24-1.16 (m, 2H), 0.92 (s, 9H).

### EXAMPLE 8

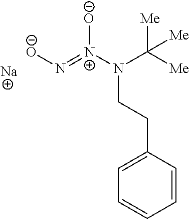 [SMILES: [Na+] ; CC(C)(C)N(CCc1ccccc1)/[N+]([O-])=N/[O-]]

### Sodium 1- [N-tert-butyl-N-(2′-phenylethyl)amino]diazen-1 -ium-1,2-diolate

The title compound was made by following the procedures described in EXAMPLE 1, substituting N-tert-butyl-N-(3′,3′-dimethylbutyl)amine for N-tert-butyl-N-propylamine.

### Activity

Compounds of the invention were evaluated for the rate of NO release using the following protocol described below. All of the examples demonstrated NO release.

The method described by Maragos, C. M, et al. Med. Chem. 1991, 34, 3242-3247, was followed. A 100 mM solution of pH 7.4 buffer was prepared by dissolving 626 mg of NaH₂PO₄.H₂O and 4155 mg of Na₂HPO₄.7H₂O in 200 mL of water. The 0.01 M solution of the individual diazeniumdiolate was prepared by dissolving the sodium diazeniumdiolate in 0.01 M sodium hydroxide solution. All solutions used were kept at room temperature. The cuvette incubator was set at 22° C. 25 μL of the 0.01 M diazeniumdiolate solution was added to a cuvette, followed by dilution with 975 μL of pH 7.4 buffer to obtain a 0.25 (25 uL of 0.01 M diluted to 1000 ul will be 0.25 mM) mM solution. The cuvette was inserted into the UV spectrometer, and its absorbance at λ=248 nm was acquired every 3 seconds.

The general first-order rate equation is:

(A−A_{∞})=(A₀−A_{∞})e^{-k!}

The method of Kezdy, F. J.; Jaz, J.; Bruylants, A. Bull. Soc. Chim. Belg. 1958, 67, 687-706, later elaborated by Schwartz, L. M.; Gelb, R. I. Anal. Chem. 1978, 50, 1592-1594, was used to analyze the data without measuring A_{∞}. Two equations are obtained when the measurement Aₜ is made at t and A_{t+Δt} is made at t+Δt. Dividing these two equations and rearranging the resultant equation, we arrive at

Aₜ=A_{t+Δt}e^{-k!}+A_{∞}(1−e^{kΔt})

Plotting Aₜ against A_{t+Δt} would yield a straight line with k=−ln(slope)/Δt, A_{∞}=intercept/(1−slope). As a result, t½=ln(2)/k.

| EXAMPLE | t½ (s) |
|---|---|
| 1 | 87 |
| 2 | 190 |
| 3 | 205 |

What is claimed is:

1. A compound of formula I: 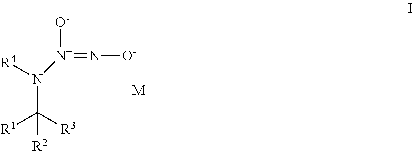
where M+ is a pharmaceutically acceptable cation;
R¹, R² and R³ are independently —C₁₋₆alkyl; and
R⁴ is —C₁₋₆alkyl, —CH₂CH═CH₂, aryl, C₃₋₈carbocycle, heteroaryl, or heterocycle;
wherein alkyl is unsubstituted or independently substituted at any carbon atom with —OH, aryl, C₃₋₈carbocycle, heteroaryl, or heterocycle, and
wherein, when R¹, R² and R³ are —CH₃, R⁴ is not —CH₃ or —CH₂CH₃.

2. A compound of claim 1, wherein
R¹, R² and R³ are independently —C₁₋₆alkyl; and
R⁴ is —C₁₋₆alkyl or —CH2CH═CH₂;
wherein alkyl is unsubstituted or independently substituted at any carbon atom with —OH or —C₆H₅.

3. A compound of claim 1, having the formula Ia, which is

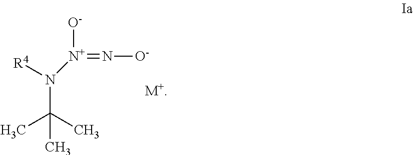

4. A compound of claim 1, wherein R⁴ is C₁₋₂alkyl that is substituted with —OH or —C₆H₅.

5. A compound of claim 1, wherein R⁴ is unsubstituted C₃₋₆alkyl.

6. A compound of claim 1, wherein R⁴ is —CH₂C₆H₅, —CH₂CH₂C₆H₅, —CH₂CH₂OH, —CH₂CH═CH₂, —CH₂CH₂CH₃, —CH₂CH₂CH₂CH₃, —CH₂CH₂CH(CH₃)₂ or —CH₂CH₂C(CH₃)₃.

7. A compound of claim 1, which is
Sodium 1-(N-tert-butyl-N-propylamino)diazen-1-ium-1,2-diolate,
Sodium 1-(N-tert-butyl-N-allylamino)diazen-1-ium-1,2-diolate,
Sodium 1-[N-tert-butyl-N-(2′-hydroxyethyl)amino]diazen-1-ium-1,2-diolate,
Sodium 1-(N-tert-butyl-N-benzylamino)diazen-1-ium-1,2-diolate,
Sodium 1-(N-tert-butyl-N-butylamino)diazen-1-ium-1,2-diolate,
Sodium 1-[N-tert-butyl-N-(3′-methylbutyl)amino]diazen-1-ium-1,2-diolate,
Sodium 1-[N-tert-butyl-N-(3′,3′-dimethylbutyl)amino]diazen-1-ium-1,2-diolate, or
Sodium 1-[N-tert-butyl-N-(2′-phenylethyl)amino]diazen-1-ium-1,2-diolate.

8. A compound of claim 1 wherein the cation is sodium or potassium.

9. A compound of claim 1 wherein the cation is sodium.

10. A method for treating hypertension in a patient comprising administering to the patient a compound of formula I: 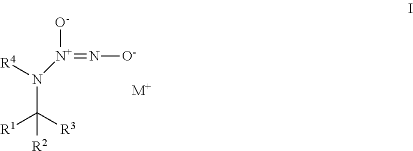
where M+ is a pharmaceutically acceptable cation;
R¹, R² and R³ are independently —C₁₋₆alkyl; and
R⁴ is —C₁₋₆alkyl or —CH₂CH═CH₂; and
wherein alkyl is unsubstituted or substituted with —OH or —C₆H₅.

11. A method of claim 11, wherein the compound is of formula la

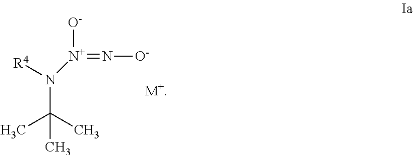

12. A method of claim 10, wherein R⁴ is C₁₋₂alkyl that is substituted with —OH or —C₆H₅.

13. A method of claim 10, wherein R⁴ is unsubstituted C₃₋₆alkyl,

14. A method of claim 10, wherein R⁴ is —CH₂C₆H₅, —CH₂CH₂C₆H₅, —CH₂CH₂OH, —CH₂CH═CH₂, —CH₂CH₂CH₃, —CH₂CH₂CH₂CH₃, —CH₂CH₂CH(CH₃)₂ or —CH₂CH₂C(CH₃)₃.

15. A method of claim 10, wherein the compound is
Sodium 1-(N-tert-butyl-N-propylamino)diazen-1-ium-1,2-diolate,
Sodium 1-(N-tert-butyl-N-allylamino)diazen-1-ium-1,2-diolate,
Sodium 1-[N-tert-butyl-N-(2′-hydroxyethyl)amino]diazen-1-ium-1,2-diolate,
Sodium 1-(N-tert-butyl-N-benzylamino)diazen-1-ium-1,2-diolate,
Sodium 1-(N-tert-butyl-N-butylamino)diazen-1-ium-1,2-diolate,
Sodium 1-[N-tert-butyl-N-(3′-methylbutyl)amino]diazen-1-ium-1,2-diolate,
Sodium 1-[N-tert-butyl-N-(3′,3′-dimethylbutyl)amino]diazen-1-ium-1,2-diolate, or
Sodium 1-[N-tert-butyl-N-(2′-phenylethyl)amino]diazen-1-ium-1,2-diolate.

16. A method of claim 10 wherein the cation is sodium or potassium.

17. A method of claim 10 wherein the cation is sodium.

18. A pharmaceutical composition comprising a compound of claim 1 and a pharmaceutically acceptable carrier.

19. A pharmaceutical composition comprising a compound of claim 7 and a pharmaceutically acceptable carrier.

20. A pharmaceutical composition comprising a compound of claim 7, a diuretic, and a pharmaceutically acceptable carrier.
